(19) United States
(12) Patent Application Publication
(10) Pub. No.: US 2023/0132044 A1
(43) Pub. Date: Apr. 27, 2023

(54) Galvanic Electrolysis Unit for Hair Follicle Ablation

(71) Applicant: Permablate Pty Ltd., Cronulla (AU)

(72) Inventor: Philip Stuart Esnouf, Melbourne (AU)

(21) Appl. No.: 17/972,667

(22) Filed: Oct. 25, 2022

Related U.S. Application Data
(60) Provisional application No. 63/271,770, filed on Oct. 26, 2021.

(51) Int. Cl. A61B 18/14; A61N 1/20

(52) U.S. Cl. CPC A61B 18/14; A61N 1/20; A61B 2018/00476

(57) ABSTRACT

A power supply unit for a direct-current galvanic electrolysis device has a housing configured to be held in a hand of a patient and having a conductive outer surface forming a first electrode that is adapted to make electrical contact with the hand of the patient while the housing is being held. In addition, the power supply unit has an electrical power source, an electrical connector supported for detachably coupling to a second electrode in the form of a needle handpiece, a regulating circuit coupled to the power source and having positive and negative output nodes. The regulating circuit positive output node is electrically coupled to the first electrode and the negative output node is electrically coupled to the connector and thereby, in use, to the second electrode of the needle handpiece.

### CROSS-REFERENCE TO RELATED APPLICATION

This application is a non-provisional filing of, and claims priority to, U.S. Application 63/271,770, filed Oct. 26, 2021, which is incorporated herein in its entirety by reference.

### FIELD

This invention relates to a direct-current galvanic electrolysis device for hair removal.

### BACKGROUND

Several treatment methods aim to permanently destroy/ablate hair-producing germinal follicles. These include electrolysis, argon laser, electro-cautery, hyfrecation, cryotherapy, and radiofrequency applications. Successful treatment requires the permanent destruction of any cells which have hair producing capacity. Any hair which needs to be plucked out—with even minimal force—is still attached to viable cells, and so the treatment application will fail.

Various methods of permanent hair removal have been in use for centuries. Galvanic electrolysis was first reported in medical literature in 1875 by ophthalmologist Charles Michel as a method for removing ingrown eyelashes. A galvanic hair remover is essentially a positive ground power supply that delivers around 0-3 milliamperes through the body. In general, a needle which is configured as the negative electrode of the electric circuit is placed along-side the hair follicle, within the dermis to complete the circuit.

Electrolysis machines known in the art utilise two leads to form the electrolysis circuit. The first of which connects a patient electrode that may be in the form of a metal electrode, with or without a conductive gel to the power source. The patient electrode may be strapped or adhered to the patient's skin. Alternatively, a flexible adhesive electrode may be used as known in the art. The power source is generally a large machine that is located on a bench or table and is commonly operated from mains electricity but may also be battery powered. From the power source a second electrical lead is provided for connection to the needle electrode as part of the operator handpiece. Many of the electrolysis machines known in the art provide high frequency alternating current as the electrolysis means, hence they do not distinguish a positive and negative electrode in the fashion of pure direct current galvanic electrolysis.

Historically, eyelid treatments have failed due to the use of topical eye-drop anaesthesia alone. Without injected local anaesthesia, the patient frequently could not tolerate the severe intra-eyelid pain of the treatment for long enough to achieve a successful permanent outcome. Sufficient treatment depth, range and duration are needed to achieve success. Under-treatment will produce tissue damage without success, and overtreatment will succeed but cause unnecessary collateral damage.

Embodiments of the present invention are designed with these issues in mind to provide successful ablation treatment.

### SUMMARY

In accordance with the present invention there is provided a power supply unit for a direct-current galvanic electrolysis device, the power supply unit comprising: a housing configured to be held in the hand of a patient, the housing having a conductive outer surface forming a first electrode that is adapted to make electrical contact with the hand of the patient while the housing is being held; an electrical power source in the form of a battery within the housing; an electrical connector supported by the housing for detachably coupling to a second electrode in the form of a needle handpiece; and a regulating circuit coupled to the power source, the regulating circuit having positive and negative output nodes; wherein the regulating circuit positive output node is electrically coupled to the first electrode and the negative output node is electrically coupled to the connector and thereby, in use, to the second electrode of the needle handpiece.

In accordance with the present invention there is also provided a direct-current galvanic electrolysis device comprising a power supply unit as defined above in combination with a needle handpiece coupled to the electrical connector of the power supply unit by way of an electrical cord.

Conveniently, the electrolysis device according to the present invention comprises two primary components: a power supply unit incorporating a first electrode and configured to be held in the hand of the patient during use, and a needle handpiece having a second electrode and configured to be manipulated by the operator. The power supply unit and the needle handpiece are coupled to one another by a flexible electrical cord.

### BRIEF DESCRIPTION OF THE DRAWINGS

Further disclosure, objects, advantages and aspects of the present invention may be better understood by those skilled in the relevant art by reference to the following description of preferred embodiments taken in conjunction with the accompanying drawings, which are given by way of illustration only and thus not limitative of the present invention, and in which:

FIG. 1 is a diagrammatic illustration of an electrolysis device according to an embodiment of the present invention;

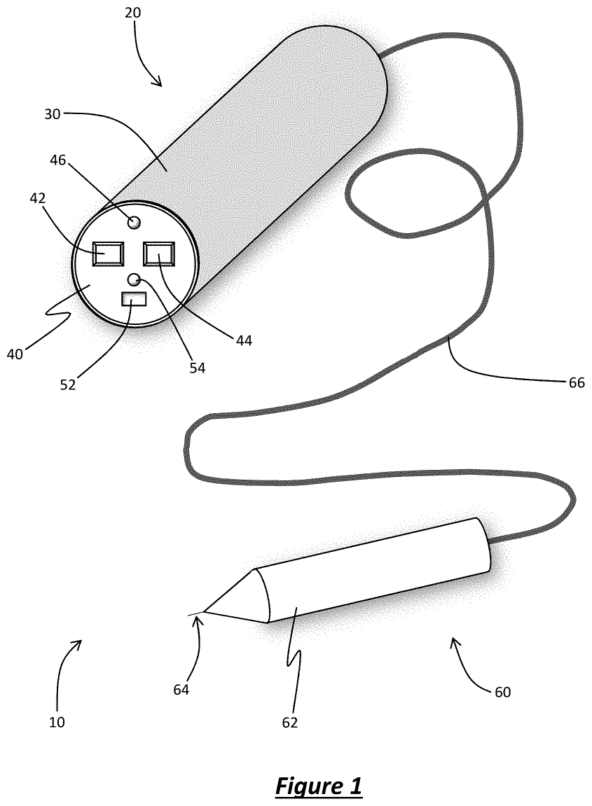

FIGS. 2A and 2B depict an operator handset and a power supply unit, respectively, according to an embodiment of the invention;

FIGS. 3A and 3B are end views of the power supply unit; and

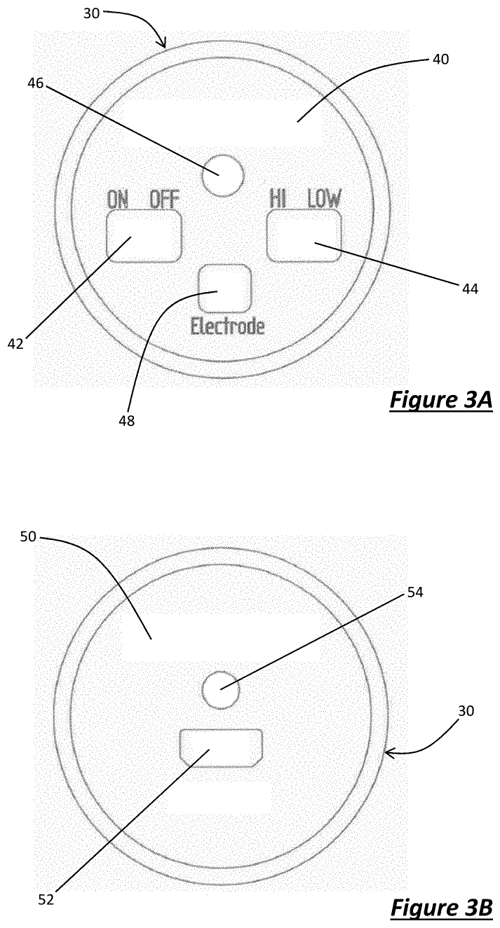

FIG. 4 is a power supply circuit diagram according to an embodiment of the invention.

### DETAILED DESCRIPTION

In the following description, where reference is made to a patient, this is understood to mean a person who is undergoing treatment for hair removal. Where reference is made to an operator, this is understood to mean the person (e.g. clinician) responsible for operating the electrolysis device according to embodiments of the invention.

The power supply unit according to a preferred embodiment provides an electrical power supply circuit to produce an output voltage, say 6-12V, suitable to perform direct-current galvanic electrolysis for the purposes of hair follicle ablation. The electrical power is derived from an in-built battery, such as a Lithium-polymer rechargeable battery.

The power supply circuit including the battery is contained within an electrically conductive metal housing that comprises the patient electrode. The tube electrode is preferably formed from stainless-steel and has a diameter such that it can comfortably be held within the patient's hand. The diameter of the tube may be in the range of approximately 15-50 mm, with the preferred diameter being approximately 29 mm. The length of the tube may be in the range from 80 mm to 120 mm, preferably about 100 mm.

The outer surface of the tube provides an electrical contact for the power supply circuit to the patient for conduction of operable current during electrolysis, in use. The current output from the voltage source contained within the electrode is limited by the circuit design associated with the power supply in a fashion that is independent of the patient's body resistance. In particular those patients who have low body resistance in the circuit. The current required for the electrolysis process may be from 0.05-3 mA. Preferably the use of an appropriate switchable circuitry limits maximum current flow to either 0.2 ma or 0.5 mA. This level of current may be selected by the clinician. Current flows from the active (negative) needle electrode to the positive pole (handheld). The strong polarity around the needle electrode produces chemical ionisation by electrolysis with production of hydroxide ions which liquefy tissue. This produces concentrated localised destruction of the hair follicle around the extremely thin needle electrode preferably 80-100 micron diameter. This occurs in 15-60 seconds. The process involves minimal pain in most bodily locations, but local anaesthesia is essential in treating eyelids.

The electrolysis machines currently known in the art, do not have the power-supply adapted to be enclosed within the patient positive electrode.

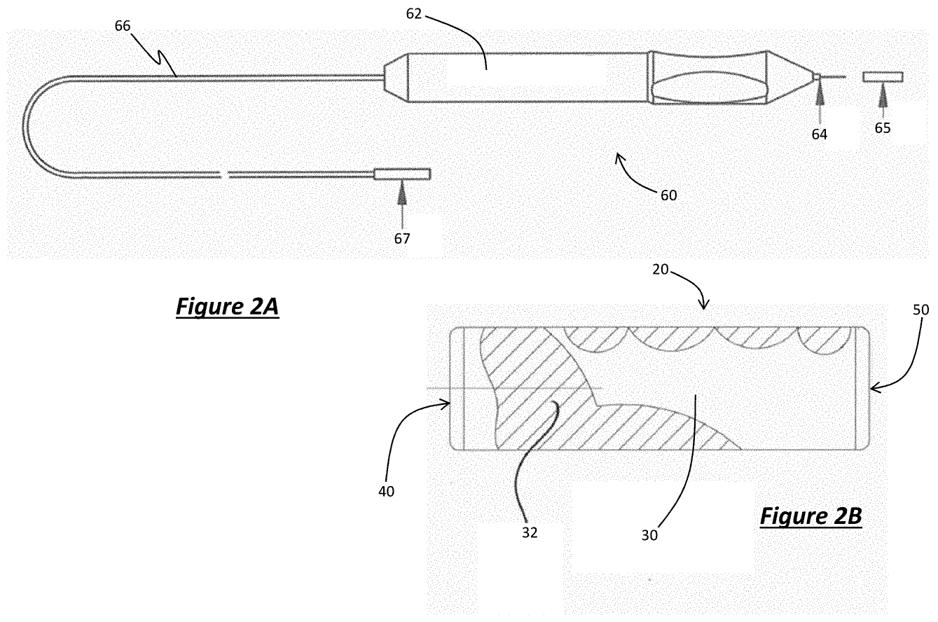

Referring to the drawings, an electrolysis device 10 according to an embodiment of the present invention comprises a power supply unit 20 and a needle electrode handpiece 60. The power supply unit 20 has a power supply housing 30, comprising a conductive tube made of an electrically conductive material such as stainless steel. The cross hatched area 32 (FIG. 2B) corresponds to the area of contact that occurs while it is held in the hand of a patient. This allows for electrical contact with the skin of the patient's hand. The electrically conductive tube is connected to the positive output of a power supply contained within the housing.

The power supply unit has first and second ends (40, 50) that may be in the form of plastic mouldings inserted in the ends of the tube. The ends of the power supply unit have control switches and electrical connector sockets for the power supply. The front moulding 40 has a socket 48 that accepts a female connector 67 on the end of an electrical cord 66 from the needle electrode handpiece 62. A switch 42 is provided to allow current to flow from the power supply. There is also a green LED indicator 46 which indicates current is available to the needle electrode. A slide switch 44 is also provided that allows current limitation to be selected at either 0.2 or 0.5 mA. The rear moulding 50 has a micro USB socket 52 to allow an internal battery of the power supply to be charged using a USB charger as known in the art. In one embodiment of the invention a Lithium-polymer rechargeable battery having a 3.7V, 800 mAh output delivery (e.g. 2.96 Wh capacity) may be employed. An LED 54 is provided that indicates the power supply is being charged by illuminating red and that the power supply is fully charged by indicating green.

The needle electrode handpiece 60 includes a gold-plated elliptical tip 64 which is blunt to allow the needle to pass along a space between the hair follicle and the dermis. Such needles are well known in the art. This needle is situated in the distal end of a pencil like plastic handle 62 and connected electrically to a light gauge wire 66 which passes out of the proximal end of the handle and terminates in the connector 67 suitable to be connected to the power supply. The needle handle and lead are packaged and presented to the operator in a sterile condition, with the tip 64 protected by a removable cap 65.

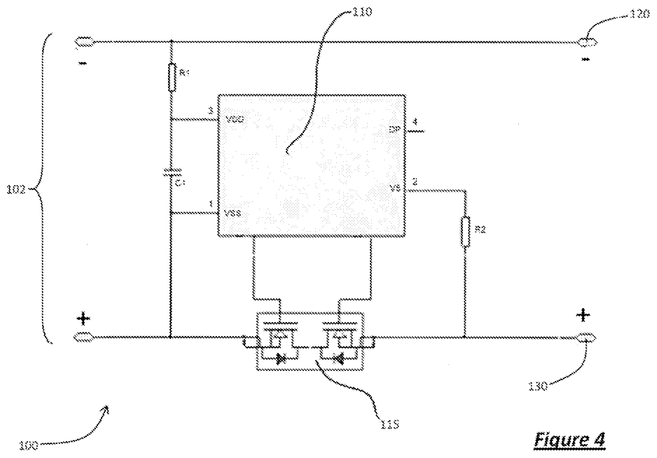

FIG. 4 is a circuit diagram of a power supply 100. The circuit board comprises electronic components including a battery and is situated within the metal tube of the power supply housing. It is held in place by the front and rear mouldings previously described. The circuit comprises a battery connected to input terminals 102 of a regulating circuit. The regulating circuit includes an integrated circuit 110 which regulates charging and output voltage of the battery. This output passes through a current limiting circuit 115, to output nodes 120, 130 that in use supply voltage to the power supply housing electrode and needle electrode respectively.

The structure and implementation of embodiments of the invention has been described by way of non-limiting example only, and many additional modifications and variations may be apparent to those skilled in the relevant art without departing from the spirit and scope of the invention described.

Any discussion of documents, devices, acts or knowledge in this specification is included to explain the context of the invention. It should not be taken as an admission that any of the material forms part of the prior art base or common general knowledge in the relevant art in Australia or elsewhere on or before the priority date of the disclosure and claims herein.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

1. A power supply unit for a direct-current galvanic electrolysis device, the power supply unit comprising:
a housing configured to be held in a hand of a patient, the housing having a conductive outer surface forming a first electrode that is adapted to make electrical contact with the hand of the patient while the housing is being held;
an electrical power source in the form of a battery within the housing;
an electrical connector supported by the housing for detachably coupling to a second electrode in the form of a needle handpiece; and
a regulating circuit coupled to the electrical power source, the regulating circuit having positive and negative output nodes;
wherein the regulating circuit positive output node is electrically coupled to the first electrode and the negative output node is electrically coupled to the electrical connector and thereby, in use, to the second electrode of the needle handpiece.

2. A direct-current galvanic electrolysis device comprising the power supply unit as defined in claim 1 in combination with the needle handpiece coupled to the electrical connector of the power supply unit by way of an electrical cord.
